(19) United States
(12) Patent Application Publication
(10) Pub. No.: US 2021/0283165 A1
(43) Pub. Date: Sep. 16, 2021

(54) Constituent combination for treating stress

(71) Applicant: Nidal Toman, Berlin (DE)

(72) Inventor: Nidal Toman, Berlin (DE)

(21) Appl. No.: 17/198,283

(22) Filed: Mar. 11, 2021

(51) Int. Cl. A61K 31/714; A61K 31/455; A61K 31/4415; A61K 31/525; A61K 31/51; A61K 33/14; A61K 47/02; A61K 31/198; A61K 31/405; A61K 31/145; A61K 31/197

(52) U.S. Cl. CPC A61K 31/714; A61K 31/455; A61K 31/4415; A61K 31/525; A61K 31/51; A61K 31/197; A61K 47/02; A61K 31/198; A61K 31/405; A61K 31/145; A61K 33/14

(57) ABSTRACT

A constituent combination for treating stress, psychological trauma or physical fatigue is dissolved in an isosmotic brine solution and includes 10-20 μmol/L methyl cobalamin, 20-30 μmol/L adenosylcobalamin, 5-8 mmol/L nicotinamide, 700-1,200 μmol/L pyridoxine, 150-250 μmol/L riboflavin-5-sodium phosphate, 70-150 μmol/L aneurin, 1-2 mmol/L magnesium chloride, 1-2 μmol/L calcium chloride and 2-3 mmol/L potassium chloride. The isosmotic brine solution is isosmotic brine solution at concentration of 0.9%.

### BACKGROUND OF INVENTION

### 1. Field of the Invention

The invention relates to a constituent combination for treating stress, psychological trauma or physical fatigue, and more particularly to a constituent combination composed of vitamin, amino acid and electrolyte.

### 2. Description of Related Art

Generally, the stress symptom and the symptoms induced by overfatigue are treated by taking a rest, which relaxes the patient, so as to restore the original state. As for the long-term therapeutic method for stress, trauma and overfatigue, the patient is suggested to take balanced diet, regular rest and moderate exercise.

These therapeutic methods can be generally classified into physical health care (but unfixable) and psychological health care. It is recognized in medical field that the feedback of mental health condition to physical health condition is an interaction. Therefore, it can be inferred that partial consciousness will act on subconsciousness, and will regulate or control the nervous system. This kind of regulation and control will bring relaxed state and promote metabolic process, and the overall sense of happiness is generated in consciousness under the effect of relaxation and metabolism, so there is very strong interaction between psychology and physiology. In order to recover the patient's normal condition from stress, psychological trauma or overfatigue faster, an appropriate constituent combination with an effective regulation and control mechanism shall be found in this interactional system for treatment.

As it is known that after cancer treatment or during treatment, the patient's recovery can be promoted by using amphetamine, and the enhancement effect of amphetamine on subjective feelings surely can induce in vivo reaction, so that the patient can recover faster. However, using amphetamine is not completely free of problems, the amphetamine addicts the patient, and the patient cannot keep judgment all the time under the effect of amphetamine.

In addition, to let the soldiers who often feel stress and psychological impact serve normally, the excitants shall not be used for treatment in a long period of time. In terms of athletes, they often fail to protect themselves during training and get hurt, but they cannot be treated with compound medicaments, because these medicaments are usually regarded as excitants, and many of them are prohibited in athletic sports.

### SUMMARY OF THE INVENTION

It is therefore one object of the invention to provide a constituent combination for treating stress, psychological trauma or physical fatigue, dissolved in an isosmotic brine solution, comprising 10-20 μmol/L Methyl cobalamin, 20-30 μmol/L Adenosylcobalamin, 5-8 mmol/L Nicotinamide, 700-1200 μmol/L Pyridoxine, 150-250 μmol/L Riboflavin-5-sodium phosphate, 70-150 μmol/L Aneurin, 1-2 mmol/L Magnesium chloride, 1-2 μmol/L Calcium chloride, and 2-3 mmol/L Potassium chloride; wherein the isosmotic brine solution refers to isosmotic brine solution at concentration of 0.9%.

The above and other objects, features and advantages of the invention will become apparent from the following detailed description taken with the accompanying drawings.

### BRIEF DESCRIPTION OF THE DRAWINGS

FIG. 1 is a first overall analysis chart according to questionnaire scoring results of all subjects for questions about physiological health in the questionnaire;

FIG. 2 is a second overall analysis chart according to questionnaire scoring results of all subjects for questions about physiological health in the questionnaire;

FIG. 3 is a first overall analysis chart according to questionnaire scoring results of all subjects for questions about mental health in the questionnaire;

FIG. 4 is a second overall analysis chart according to questionnaire scoring results of all subjects for questions about mental health in the questionnaire;

FIG. 5 is a first questionnaire scoring result analysis chart of quality of life filled by all subjects before the invention was used and four weeks after use;

FIG. 6 is a second questionnaire scoring result analysis chart of quality of life filled by all subjects before the invention was used and four weeks after use;

FIG. 7 is a first questionnaire scoring result analysis chart of satisfaction at health condition filled by all subjects before the invention was used and four weeks after use;

FIG. 8 is a second questionnaire scoring result analysis chart of satisfaction at health condition filled by all subjects before the invention was used and four weeks after use;

FIG. 9 is a first questionnaire scoring (negative values) result analysis chart of influence of physiological pains on daily life filled by all subjects before the invention was used and four weeks after use;

FIG. 10 is a second questionnaire scoring (negative values) result analysis chart of influence of physiological pains on daily life filled by all subjects before the invention was used and four weeks after use;

FIG. 11 is a first questionnaire scoring (negative values) result analysis chart for how much medical assistance is required in daily life filled by all subjects before the invention was used and four weeks after use;

FIG. 12 is a second questionnaire scoring (negative values) result analysis chart for how much medical assistance is required in daily life filled by all subjects before the invention was used and four weeks after use;

FIG. 13 is a first questionnaire scoring result analysis chart of satisfaction at daily life filled by all subjects before the invention was used and four weeks after use;

FIG. 14 is a second questionnaire scoring result analysis chart of satisfaction at daily life filled by all subjects before the invention was used and four weeks after use;

FIG. 15 is a first questionnaire scoring result analysis chart of felt significance of daily life filled by all subjects before the invention was used and four weeks after use;

FIG. 16 is a second questionnaire scoring result analysis chart of felt significance of daily life filled by all subjects before the invention was used and four weeks after use;

FIG. 17 is a first questionnaire scoring result analysis chart of mental concentration ability filled by all subjects before the invention was used and four weeks after use;

FIG. 18 is a second questionnaire scoring result analysis chart of mental concentration ability filled by all subjects before the invention was used and four weeks after use;

FIG. 19 is a first questionnaire scoring result analysis chart for the felt degree of adequate energy in daily life filled by all subjects before the invention was used and four weeks after use;

FIG. 20 is a second questionnaire scoring result analysis chart for the felt degree of adequate energy in daily life filled by all subjects before the invention was used and four weeks after use;

FIG. 21 is a first questionnaire scoring result analysis chart of physical performance (strength) condition filled by all subjects before the invention was used and four weeks after use;

FIG. 22 is a second questionnaire scoring result analysis chart of physical performance (strength) condition filled by all subjects before the invention was used and four weeks after use;

FIG. 23 is a first questionnaire scoring result analysis chart of outgoing mobility filled by all subjects before the invention was used and four weeks after use;

FIG. 24 is a second questionnaire scoring result analysis chart of outgoing mobility filled by all subjects before the invention was used and four weeks after use;

FIG. 25 is a first questionnaire scoring result analysis chart of satisfaction at sleep filled by all subjects before the invention was used and four weeks after use;

FIG. 26 is a second questionnaire scoring result analysis chart of satisfaction at sleep filled by all subjects before the invention was used and four weeks after use;

FIG. 27 is a first questionnaire scoring result analysis chart of ability for day-to-day activity filled by all subjects before the invention was used and four weeks after use;

FIG. 28 is a second questionnaire scoring result analysis chart of ability for day-to-day activity filled by all subjects before the invention was used and four weeks after use;

FIG. 29 is a first questionnaire scoring result analysis chart of satisfaction at working ability filled by all subjects before the invention was used and four weeks after use;

FIG. 30 is a second questionnaire scoring result analysis chart of satisfaction at working ability filled by all subjects before the invention was used and four weeks after use;

FIG. 31 is a first questionnaire scoring result analysis chart of self-satisfaction filled by all subjects before the invention was used and four weeks after use;

FIG. 32 is a second questionnaire scoring result analysis chart of self-satisfaction filled by all subjects before the invention was used and four weeks after use;

FIG. 33 is a first questionnaire scoring result analysis chart of frequency of negative emotion filled by all subjects before the invention was used and four weeks after use; and

FIG. 34 is a second questionnaire scoring result analysis chart of frequency of negative emotion filled by all subjects before the invention was used and four weeks after use.

### DETAILED DESCRIPTION OF THE INVENTION

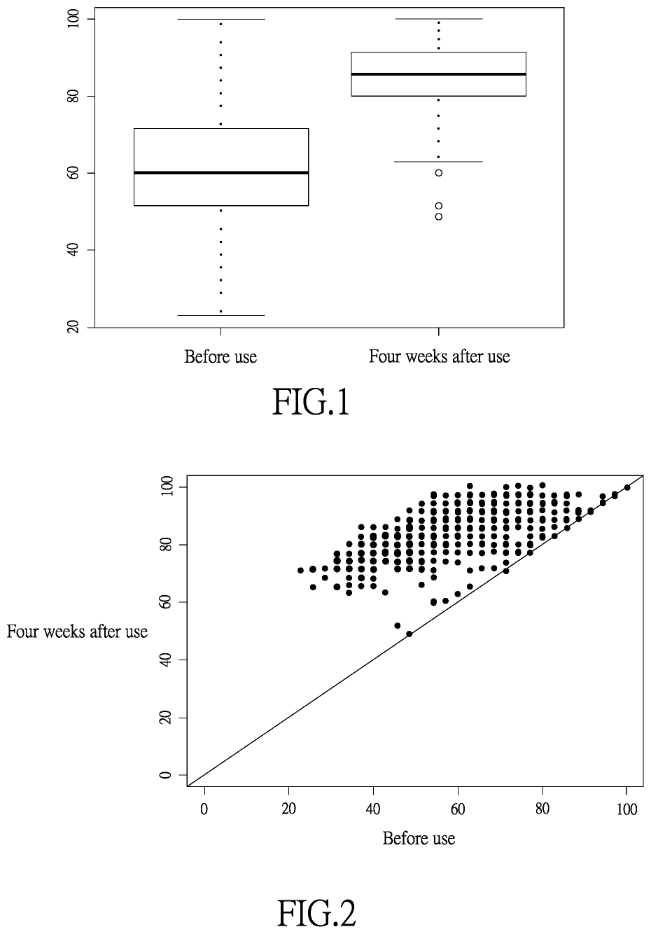

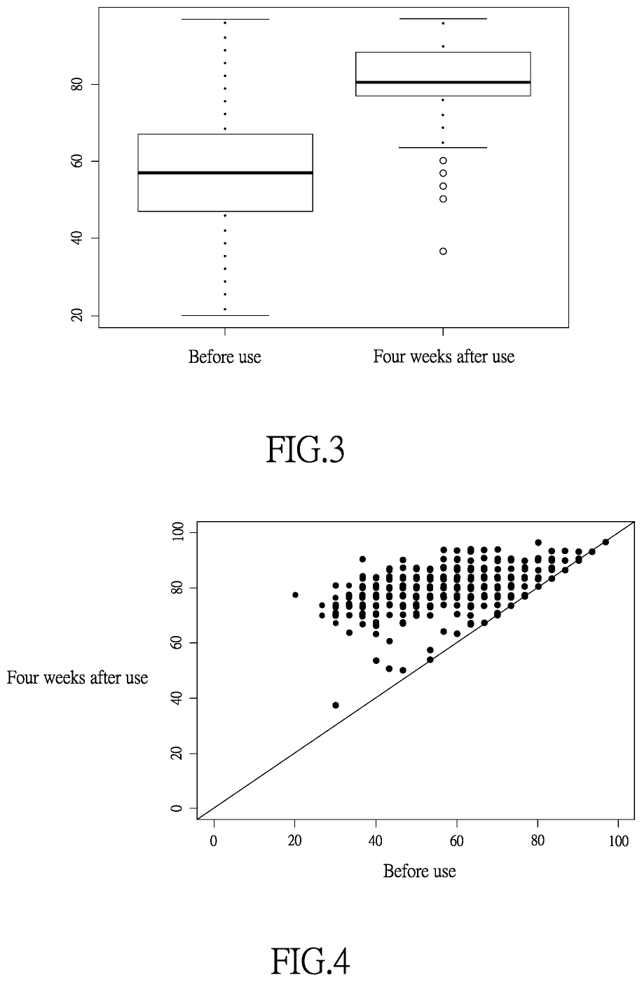

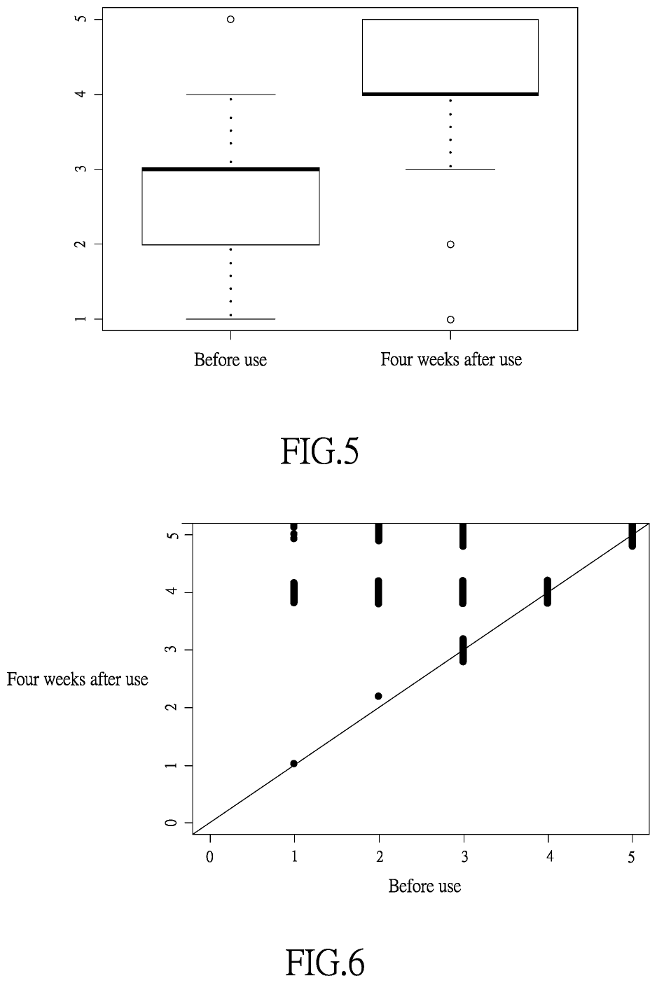

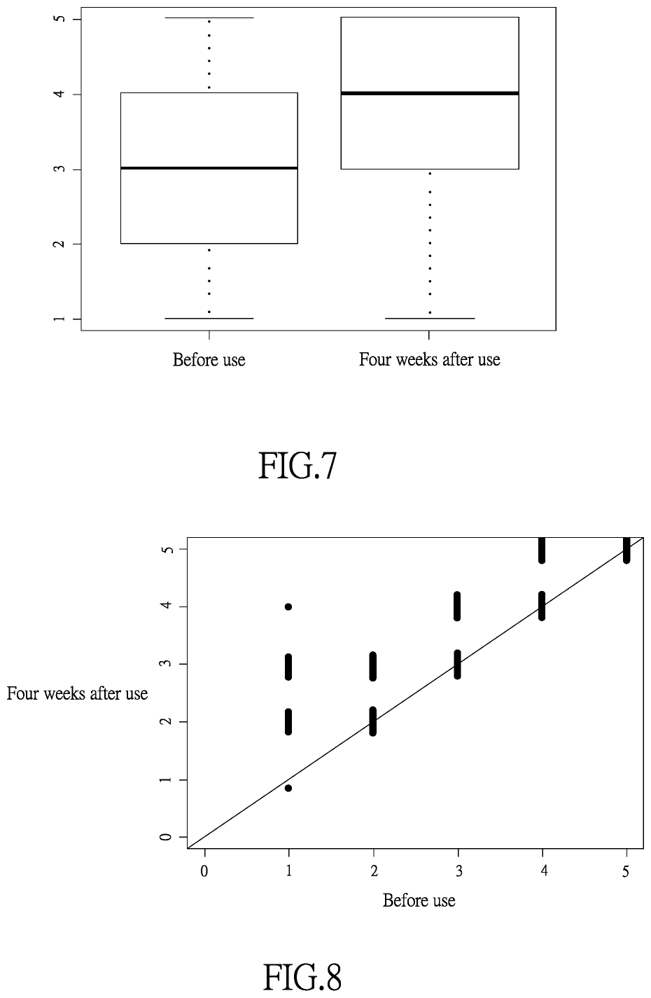

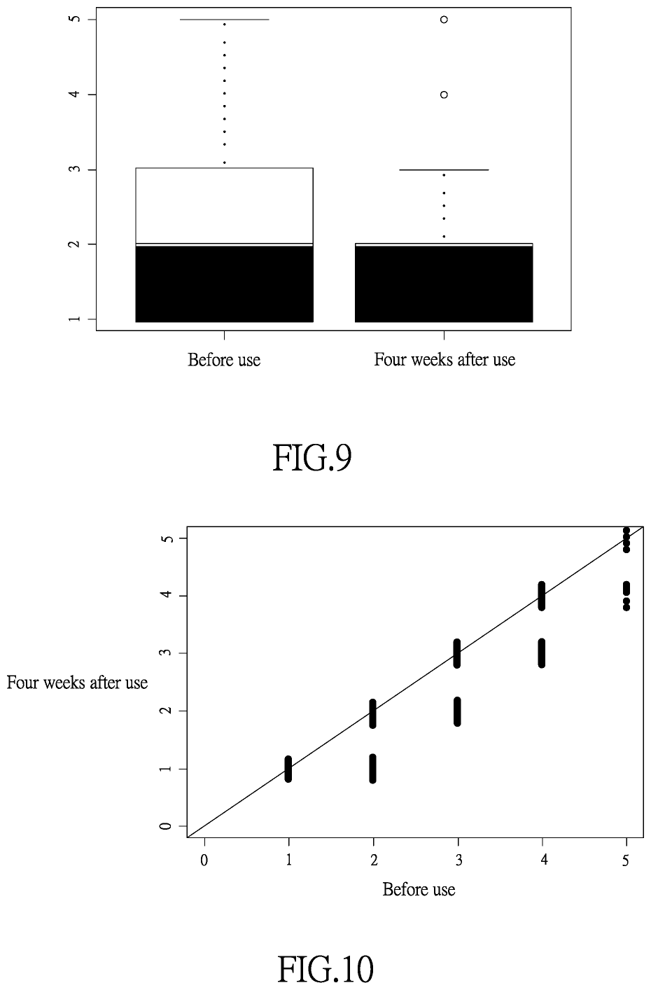

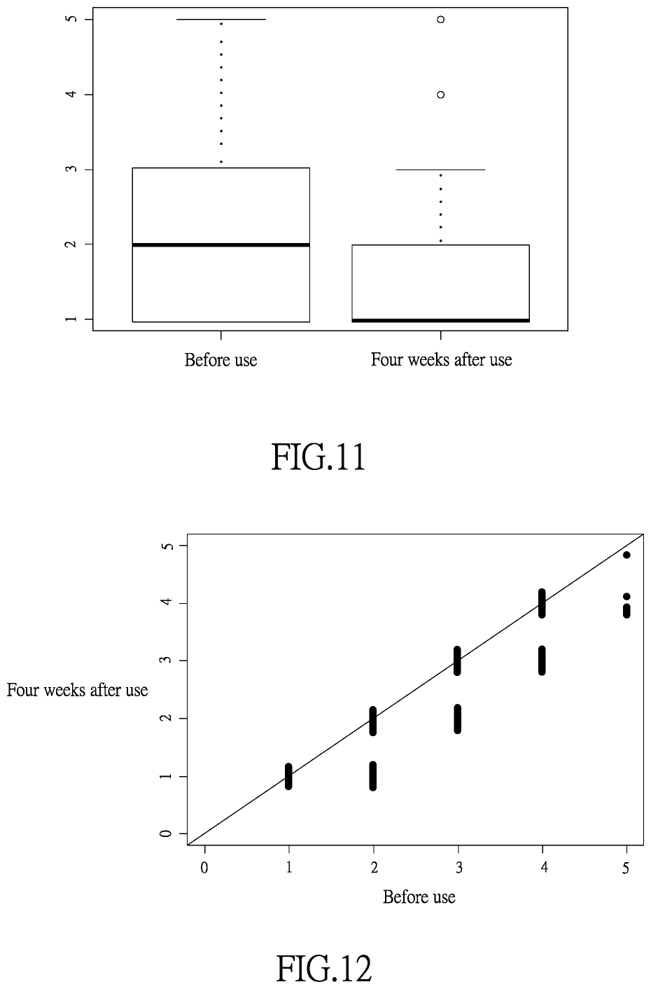

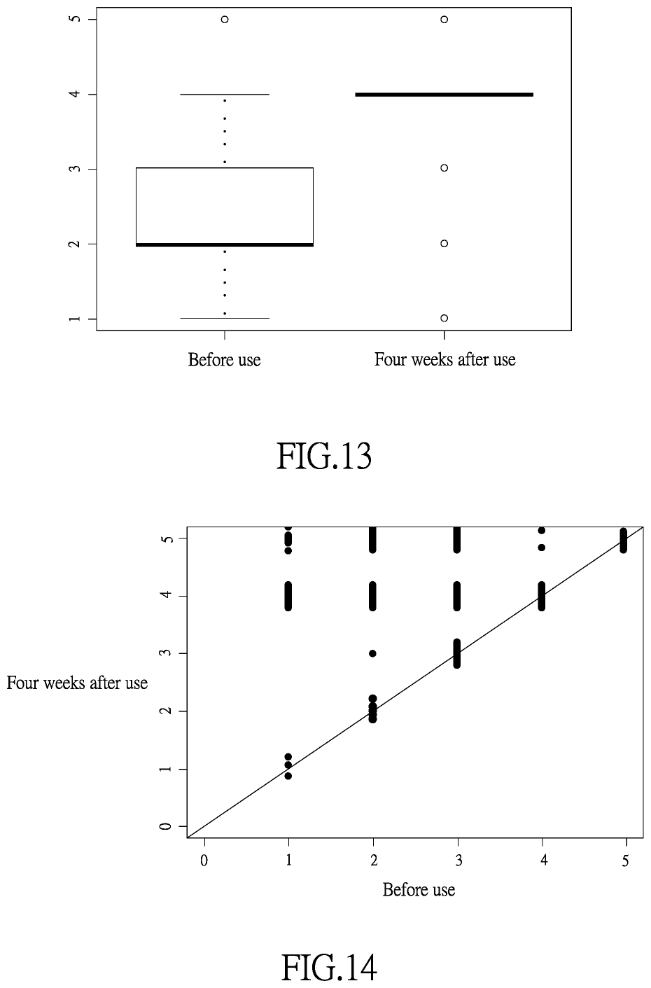

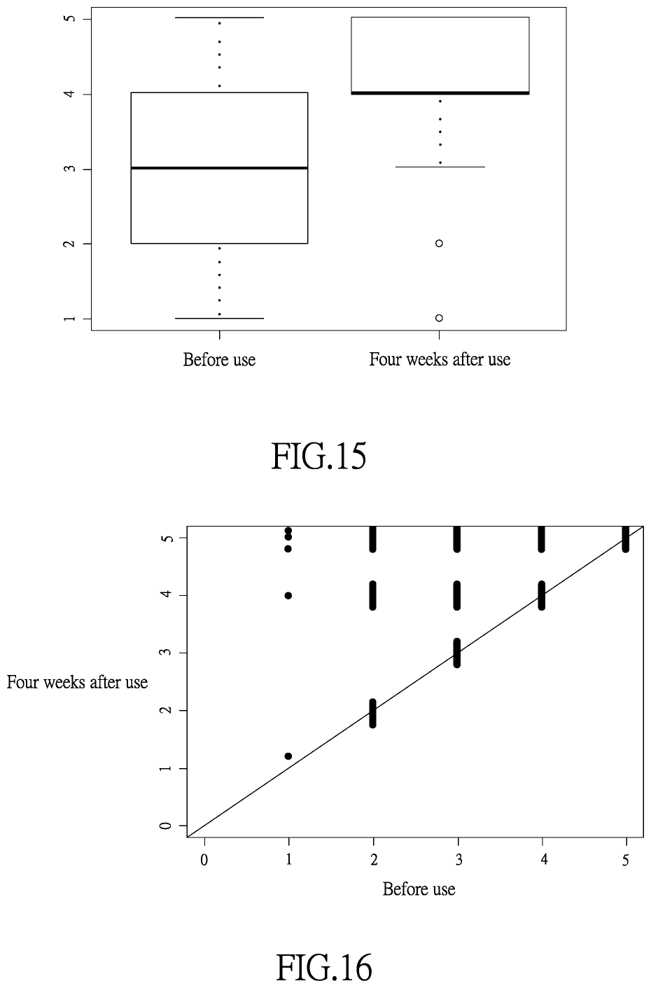

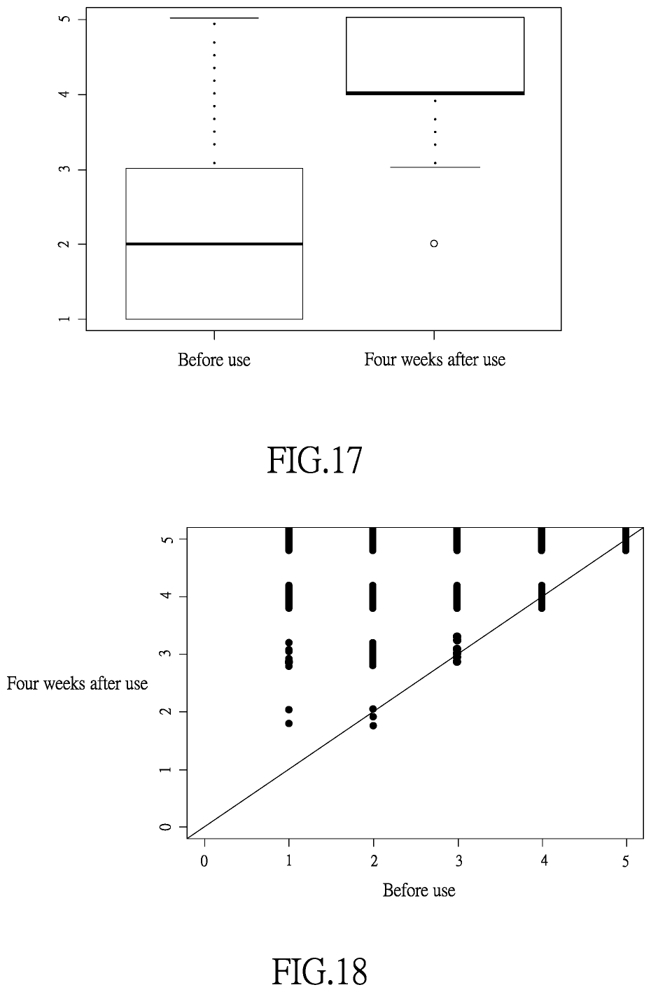

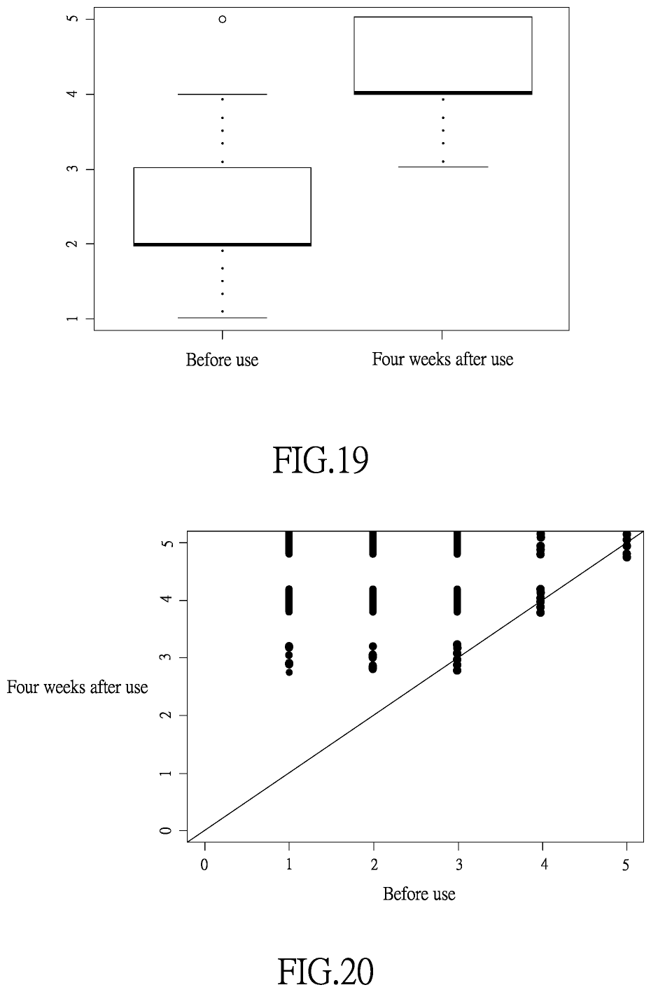

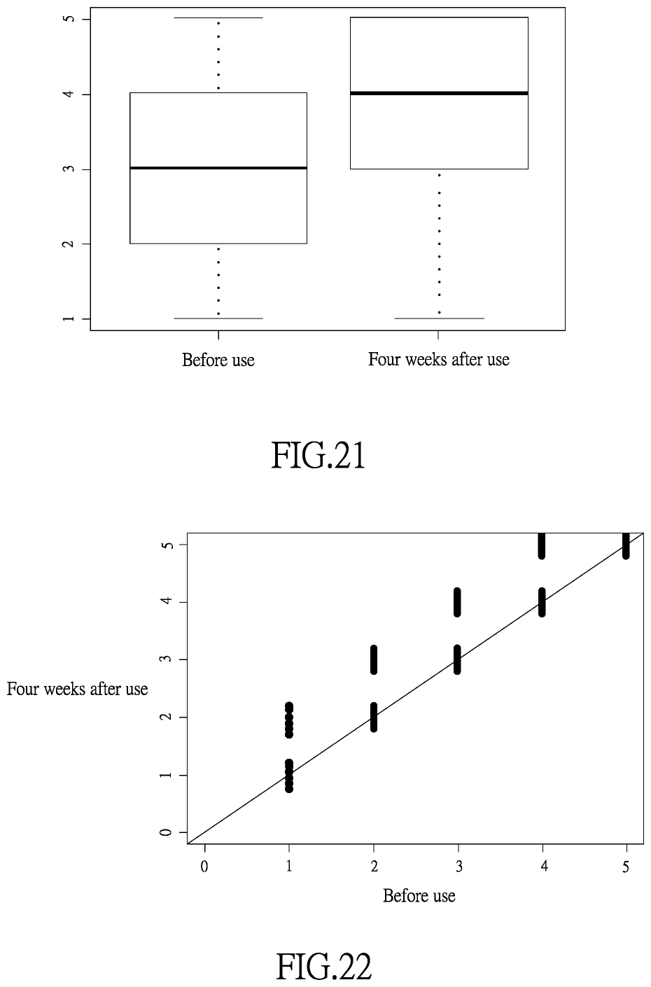

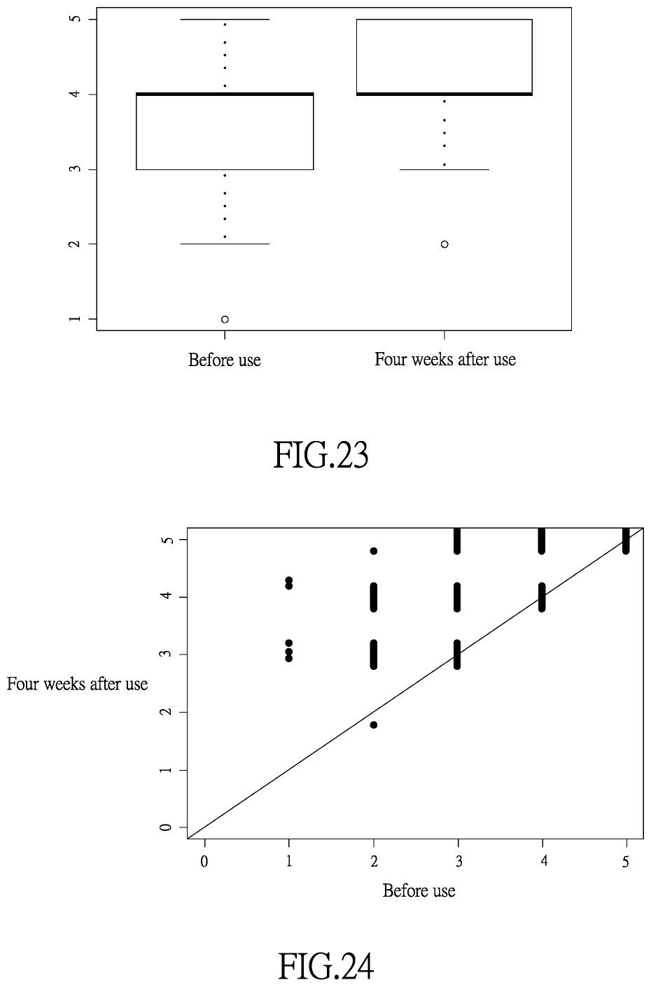

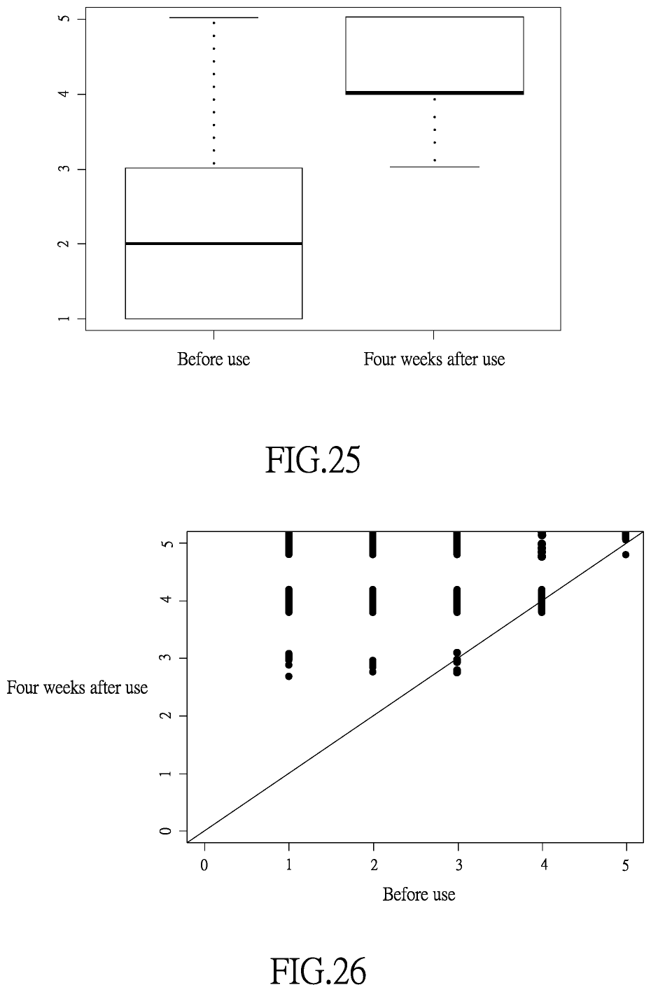

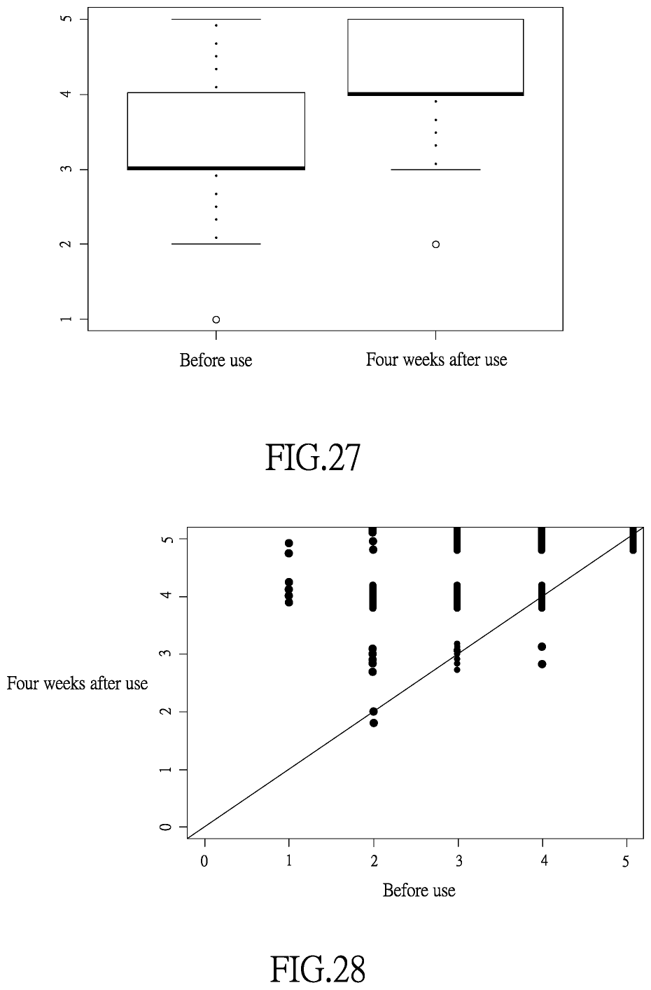

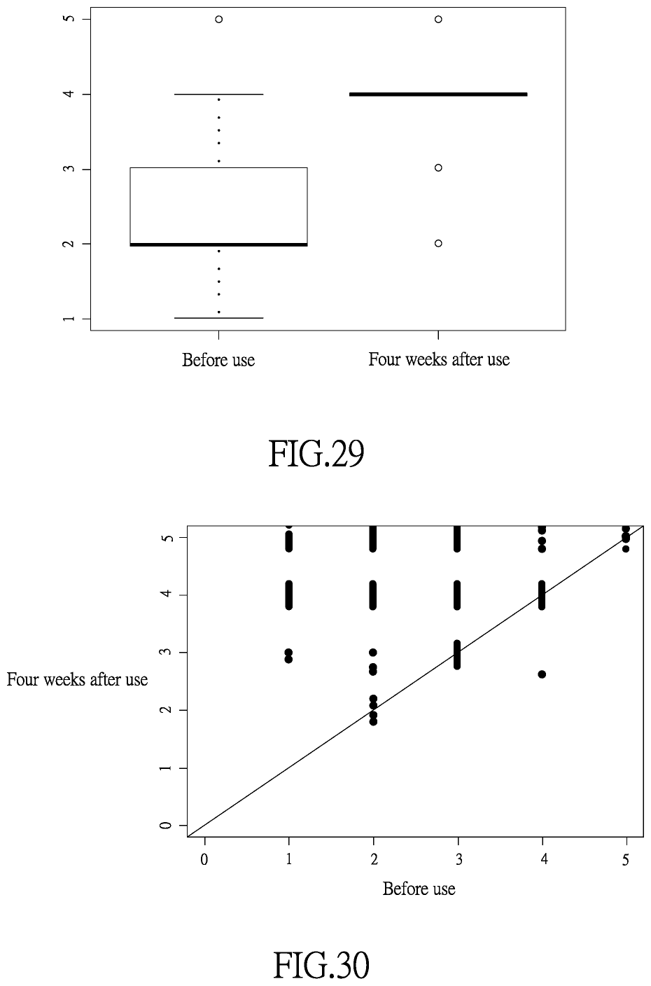

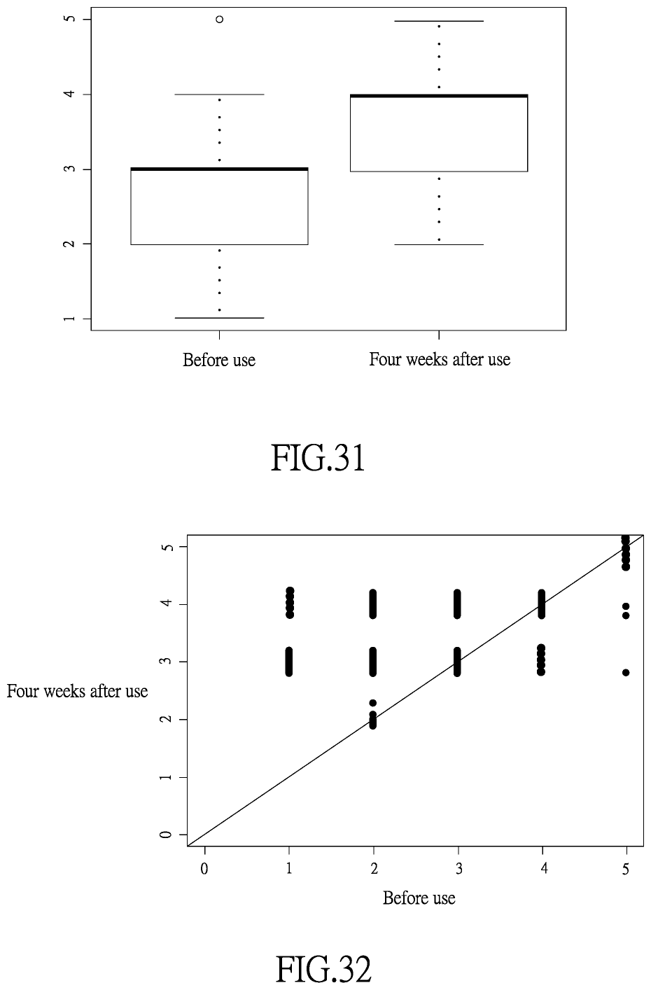

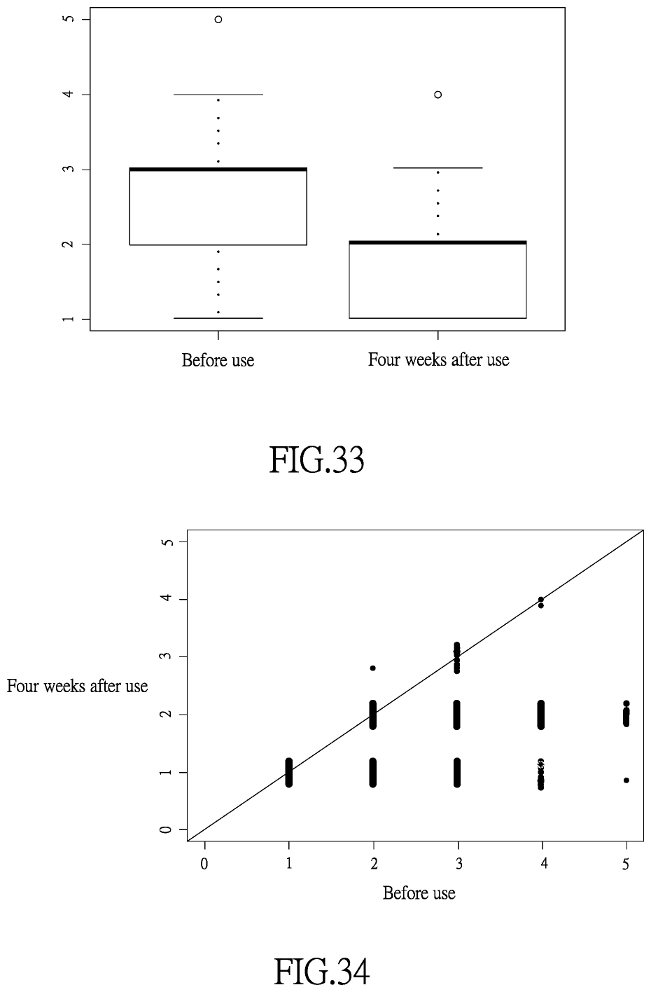

Embodiment 1, please refer to FIG. 1 to FIG. 34 for a constituent combination for treating stress, psychological trauma or physical fatigue. The constituent combination is dissolved in an isosmotic brine solution and comprises 10-20 μmol/L methyl cobalamin, 20-30 μmol/L adenosylcobalamin, 5-8 mmol/L nicotinamide, 700-1,200 μmol/L pyridoxine, 150-250 μmol/L riboflavin-5-sodium phosphate and 70-150 μmol/L aneurin and electrolyte 1-2 mmol/L magnesium chloride, 1-2 μmol/L calcium chloride and 2-3 mmol/L potassium chloride; wherein the isosmotic brine solution is isosmotic brine solution at concentration of 0.9%.

The constituent combination can contain the first other effective constituents: 4-8 mmol/L phenylalanine, 5-10 mmol/L N-acetyl-L-tyrosine, 30-50 mmol/L L-arginine, 20-40 mmol/L taurine, 30-70 mmol/L glycine, 10-30 mmol/L L-carnitine, 2-5 mmol/L L-carnosine and 30-50 mmol/L L-lysine to form a first other effective constituent combination.

The constituent combination can contain the second other effective constituents: 10-30 mmol/L L-isoleucine, 20-40 mmol/L L-leucine, 10-30 mmol/L L-lysine hydrochloride, 5-15 mmol/L L-methionine, 10-25 mmol/L L-phenylalanine, 10-25 mmol/L L-taurine, 10-30 mmol/L L-threonine, 2-5 mmol/L L-tryptophan and 20-40 mmol/L L-valine to form a second other effective constituent combination.

The concentrations of the constituent combination, the first other effective constituent combination and the second other effective constituent combination are adjusted to 0.5-2 mmol/L using sodium hydroxide and hydrochloric acid, and the pH is 6.5-7.5.

The first other effective constituent combination can be used in the drugs for treating such stress-related diseases as fatigue syndrome, aprosexia, tiredness and chronic fatigue syndrome. The second other effective constituent combination can be used in the drugs for treating overfatigue symptoms induced by alcohol or drug abuse; or in the drugs for treating overfatigue symptoms induced by strenuous exercise or muscular training.

Generally, the stress symptoms and the symptoms induced by overfatigue are treated by taking a rest, which relaxes the patient, so as to recover the original state. In terms of long-term therapeutic method for stress, trauma and overfatigue, the patient is suggested to take balanced diet, regular rest and moderate exercise. These therapeutic methods can be generally classified into physical health care (but unfixable) and psychological health care. It is recognized in medical field that the feedback of mental health condition to physical health condition is an interaction. Therefore, it is inferred that partial consciousness will act on subconsciousness, and can regulate or control nervous system. This kind of regulation and control will bring relaxed state and promote metabolic process. An overall sense of happiness will be generated in consciousness under the effect of relaxation and metabolism, so there is very strong interaction between psychology and physiology.

The patient cannot remain in the supposed healthy life state all the time due to external environment, so a therapeutic method had better be provided for the patient in unhealthy life stage, this therapeutic method will not be addictive or damage the patient's judgment, and it will not be regarded as the prohibited excitants, and it is applicable to long-term treatment.

According to the results of comprehensive research reports on vitamin and the effect thereof, taking vitamin in a certain period can remedy the subnutrition induced by diet or life style. These studies are based on the action principle of pure substance or single effective substance and extensively used in Western medicine. The traditional Chinese medicine is based on mixtures, these mixtures are derived from long-term practical observation and spread through documents or from mouth to mouth.

In addition, according to research findings, the human intestinal tract has a key effect on the overall sense of happiness, because the serotonin can be synthesized in intestinal tract. The serotonin is a nerve conduction substance, the internal serotonin level has considerable influence on the patient's subjective sense of happiness. If the serotonin level in human intestinal tract is lower than ordinary level, that will induce continuous depressive emotion, this is why the balanced diet and moderate exercise which can promote intestinal peristalsis have an important effect on the generation of the sense of happiness.

Therefore, in order to recover the patient to normal condition from stress, psychological trauma or overfatigue faster, the invention is a constituent combination composed of vitamin, amino acid and electrolyte, the constituent combination with an effective regulation and control mechanism can be used in this interactional system for treating the physiological or psychological symptoms induced by stress, psychological trauma or overfatigue.

The constituent combination can promote the formation of serotonin in intestinal tract. When the internal serotonin level rises, the patient feels stronger sense of happiness. This sense of happiness can increase physical activity, the physical metabolism is promoted by natural purification, so as to remove the end product of physical tissue metabolism, and self-reinforcement is achieved by this cycle to effectively relieve the stress symptom, psychological trauma and overfatigue. According to experimental observation results of long-term usage of the constituent combination for soldiers and athletes, the single effect of vitamin, i.e. singly taking one substance, is different from the effect of taking a specific mixture.

The constituent combination is a necessary effective constituent combination in the first other effective constituent combination or the second other effective constituent combination, because when they are used together, the effect of the first other effective constituents or the second other effective constituents can be increased or enhanced obviously. A questionnaire designed by the WHO is used to investigate more than 600 patients for overall feeling before and after the invention is used, the result shows the first other effective constituents and the second other effective constituents are effective.

When the first other effective constituents of the first other effective constituent combination are used with the constituent combination, stable subjective emotion can be provided, so the first other effective constituents have assistant effect on treating stress symptom and psychological trauma. The typical manifestations of intense stress relief include cognitive function enhancement (attention, perception), mental and physical senses of satisfaction, physical status enhancement, stress symptom relief (e.g. alopecia, gastralgia, sleep disorder).

When the second other effective constituents of the second other effective constituent combination and the constituent combination are used together, the overfatigue can be recovered faster, so as to alleviate the symptom of overfatigue after high pressure training or strenuous physical exercise. As the products of exchange of substance, such as the lactic acid resulted from muscular high-strength load or the fusel oil which induces strong discomfort, headache and migraine after excessive drinking, especially a slight amount of methanol, can be removed faster, the internal blood vessels can be relaxed. This transport process can relax the internal poisoned physical tissues, and the relaxation promotes blood circulation, so that the poison materials can be removed faster, and the body recovers faster. Therefore, the second other effective constituents have assistant effect on treating the overfatigue symptom induced by intense physical load or alcohol and drug abuse.

The therapeutic method used in the invention is a branch “psychophysiology” of medicine. The psychophysiology is a science which uses psychological, physiological and biological methods to discuss the psychology-physiology interaction and the effect thereof. The psychological condition is an objective parameter of the patient's body, and the physiological condition is an objective parameter of psychology. However, the mentality can be “measured” only by questionnaire survey of the patient, i.e. the patient's subjective feeling state. On the contrary, the physiological condition can be judged according to different objective measured data. Wherein there is no objective parameter for describing the patient's subjective feeling so far.

The effect of two injection solutions (isosmotic brine solution) is investigated by using a prospective method, which are the injection solution containing the first other effective constituent combination and the injection solution containing the second other effective constituent combination. Wherein the amount of the first other effective constituent combination or the second other effective constituent combination is obtained by multiplying the concentration by the dose of injection solution, and it is intravenously injected into human body at a speed of 3 drips/sec, 250 ml/drip, the administration time is about 30 minutes.

The patients under treatment were asked to fill in the WHO questionnaire WHOQOL-BREF, and investigated for their subjective feelings. When the treatment process began, the patients filled in the questionnaires before the first intravenous injection, and the patients were intravenously injected once every week in the next four weeks, the patients' subjective feelings were investigated by questionnaires after the treatment process was finished. The questions (15 questions) of WHO questionnaire WHOQOL-BREF involved the quality of life and general health, the latter one included physiological health and mental health.

There were two groups investigated. Group 1 had 637 participants, treated with the injection solution containing the first other effective constituent combination. The 637 participants included 169 females and 468 males, the average age was 47 (minimum age was 45, maximum age was 71). Group 2 had 829 participants, treated with injection solution containing the second other effective constituent combination. The 829 participants included 400 females and 429 males, the average age was 30 (minimum age was 21, maximum age was 51). FIGS. 1 to 34 show the questionnaire survey results before treatment and after treatment. Generally speaking, the perception of quality of life is improved.

While the invention has been described in terms of preferred embodiments, those skilled in the art will recognize that the invention can be practiced with modifications within the spirit and scope of the appended claims.

What is claimed is:

1. A constituent combination for treating stress, psychological trauma or physical fatigue, dissolved in an isosmotic brine solution, comprising 10-20 μmol/L Methyl cobalamin, 20-30 μmol/L Adenosylcobalamin, 5-8 mmol/L Nicotinamide, 700-1200 μmol/L Pyridoxine, 150-250 μmol/L Riboflavin-5-sodium phosphate, 70-150 μmol/L Aneurin, 1-2 mmol/L Magnesium chloride, 1-2 μmol/L Calcium chloride, and 2-3 mmol/L Potassium chloride; wherein the isosmotic brine solution is isosmotic brine solution at concentration of 0.9%.

2. The constituent combination of claim 1, comprising 4-8 mmol/L Phenylalanine, 5-10 mmol/L N-acetyl-L-tyrosine, 30-50 mmol/L L-arginine, 20-40 mmol/L Taurine, 30-70 mmol/L Glycine, 10-30 mmol/L L-carnitine, 2-5 mmol/L L-carnosine and 30-50 mmol/L L-lysine.

3. The constituent combination of claim 1, comprising 10-30 mmol/L L-isoleucine, 20-40 mmol/L L-leucine, 10-30 mmol/L L-lysine hydrochloride, 5-15 mmol/L L-methionine, 10-25 mmol/L L-phenylalanine, 10-25 mmol/L L-taurine, 10-30 mmol/L L-threonine, 2-5 mmol/L L-tryptophan and 20-40 mmol/L L-valine.

4. The constituent combination of claim 1, wherein the pH of said constituent combination is 6.5-7.5, the pH is adjusted by using sodium hydroxide and hydrochloric acid, the concentration of the constituent combination is 0.5-2 mmol/L.

5. The constituent combination of claim 2, wherein the pH of said constituent combination is 6.5-7.5, the pH is adjusted by using sodium hydroxide and hydrochloric acid, the concentration of the constituent combination is 0.5-2 mmol/L.

6. The constituent combination of claim 3, wherein the pH of said constituent combination is 6.5-7.5, the pH is adjusted by using sodium hydroxide and hydrochloric acid, the concentration of the constituent combination is 0.5-2 mmol/L.

7. The constituent combination of claim 2, wherein said constituent combination can be used in the drugs for treating such stress-related diseases as fatigue syndrome, aprosexia, tiredness and chronic fatigue syndrome.

8. The constituent combination of claim 4, wherein said constituent combination can be used in the drugs for treating such stress-related diseases as fatigue syndrome, aprosexia, tiredness and chronic fatigue syndrome.

9. The constituent combination of claim 3, wherein said constituent combination can be used in the drugs for treating overfatigue symptom induced by alcohol or drug abuse.

10. The constituent combination of claim 4, wherein said constituent combination can be used in the drugs for treating overfatigue symptom induced by alcohol or drug abuse.

11. The constituent combination of claim 3, wherein said constituent combination can be used in the drugs for treating overfatigue symptom induced by strenuous exercise or muscular training.

12. The constituent combination of claim 4, wherein said constituent combination can be used in the drugs for treating overfatigue symptom induced by strenuous exercise or muscular training.
